(19) United States
(12) Patent Application Publication
(10) Pub. No.: US 2026/0232109 A1
(43) Pub. Date: Aug. 13, 2026

(54) MEDICAL CABINET WITH SELECTIVELY ACCESSIBLE STORAGE CONTAINERS

(71) Applicant: CareFusion 303, Inc., San Diego, CA (US)

(72) Inventors: Jaewoo Kim, San Diego, CA (US); Colburn Leonard Jones, Butler, PA (US); Matthew Alvin Tupps, Ypsilanti, MI (US)

(21) Appl. No.: 19/047,688

(22) Filed: Feb. 7, 2025

(51) Int. Cl. A47B 67/02; A61G 12/00; G16H 20/13

(52) U.S. Cl. CPC A47B 67/02; A61G 12/001; G16H 20/13; A47B 2067/025

(57) ABSTRACT

A cabinet for storing medication and/or medical equipment includes: a housing including a cavity; at least one drawer mounted in the cavity, the drawer including at least one open container mounted in the drawer with an internal compartment and an open upper end, and at least one lidded container mounted in the drawer with an internal compartment and a lid. The lid is attached to the at least one lidded container via a lid mechanism and is configured to move the lid between an open position and a closed position. The cabinet further contains a controller operatively connected to the lid mechanism.

### FIELD OF THE INVENTION

The present disclosure generally relates to a device for storing and selectively permitting access to pharmaceuticals and other medical supplies.

### BACKGROUND

Hospitals and other health care settings (e.g., skilled nursing facilities, outpatient facilities, retirement homes and communities, etc.) have a need to provide organized storage for medications and supplies. It is desirable for care givers to be able to quickly and efficiently find such medications and supplies. In many instances, such containers are stored in a local automated dispensing machine (ADM). In many instances the ADM includes one or more drawers that slide between open and closed positions to provide access thereto.

Such drawers currently come in a variety of configurations. In some instances, a drawer without any additional sub-divisions is sufficient. In other instances, it may be desirable to sub-divide the drawer into smaller sections using dividing walls or individual containers. Such sub-divisions may greatly increase the organization of medications or supplies within the drawer with minimal loss of storage space. Some such drawers are configured with many open, often identical, containers with little or no free space other than that within the open containers.

Certain medications such as narcotics and controlled substances require additional levels of security while still making the medications available to caregivers. One method of accomplishing this is to use lidded containers, which are designed so that the lid can only be opened when the container is connected to a power source and processor that can send the appropriate digital commands to permit opening. The lidded container may be filled with a medication in the pharmacy and may then have the lid closed. The secure containers are installed within the drawers. The ADM can provide power and can send the commands to open the lid of the container, and can also regulate the opening of the drawers. Software of the ADM is configured to open the drawer and/or the lid of the container only after certain requirements are met, such as verification that the individual accessing the container is authorized to do so.

Selectively accessible containers have taken a number of different forms. Exemplary configurations are shown in U.S. Patent Nos. 9,687,075; 10,029,829 and 10,435,918, each of which is hereby incorporated by referenced herein in its entirety. Typically, medications are stored in a vial, jar, bottle, or the like that is stored within the container, or in other instances the medications are loosely stored within the container. In either instance the medications are secure, as access is prohibited to those without appropriate clearance/permission (i.e., the lid of the container will not open without such clearance/permission).

Although ADMs with secure containers may be adequate for many medications, there may be instances in which a cabinet includes some regulated medications that require additional levels of security and other medications that do not require secure containers. As such, it may be desirable to provide additional configurations for secure dispensing of medications in hospital settings.

### SUMMARY

As a first aspect, embodiments of the invention are directed to a cabinet for holding and dispensing items including: a housing including a cavity; at least one drawer mounted in the cavity, the drawer including at least one open container mounted in the drawer with an internal compartment and an open upper end, and at least one lidded container mounted in the drawer with an internal compartment and a lid. The lid is attached to the at least one lidded container via a lid mechanism and is configured to move the lid between an open position and a closed position. The cabinet further contains a controller operatively connected to the lid mechanism.

As a second aspect, embodiments of the invention are directed to a drawer for a cabinet for holding and dispensing items. The drawer includes: a frame including a floor and front wall; at least one open container mounted in the drawer with an internal compartment, an open upper end, and a floor; at least one lidded container mounted in the drawer with an internal compartment, a lid, and a floor, wherein the lid is attached to the lidded container via a lid mechanism; and a controller operatively connected to the lid mechanism. The floors of each of the at least one open container and the at least one lidded container are substantially coplanar.

As a third aspect, embodiments of the invention are directed to a cabinet for holding and dispensing items including: a housing including a cavity; at least one drawer mounted in the cavity, the drawer including at least one open container mounted in the drawer with an internal compartment, a local controller, and an open upper end. No containers in the drawer are lidded containers. The cabinet further contains a main controller operatively connected to the lid mechanism.

### BRIEF DESCRIPTION OF THE FIGURES

FIG. 1 is a schematic perspective view of an automated dispensing machine according to some embodiments of the invention.

FIG. 2 is a schematic perspective view of a drawer of an automated dispensing machine according to some embodiments of the invention.

FIG. 3 is a schematic perspective view of a drawer of an automated dispensing machine according to some embodiments of the invention.

FIG. 4 is a schematic perspective view of an open container according to some embodiments of the invention.

FIG. 5 is a schematic perspective view of a lidded container according to some embodiments of the invention.

FIG. 6 is a schematic perspective view of a lidded container that includes a container locking mechanism according to some embodiments of the invention.

FIG. 7 is a schematic perspective view of an open container that includes a port according to some embodiments of the invention.

FIG. 8 is a schematic perspective view of a third lidded container that includes a container locking mechanism according to some embodiments of the invention.

FIG. 9 is a schematic perspective view of the bottom of lidded a container that includes a port according to some embodiments of the invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout and different embodiments of like elements can be designated using a different number of superscript indicator apostrophes (e.g., 10', 10'', 10''') or using different letters in conjunction with the same base reference number (e.g., 10a, 10b, 10c).

In the figures, certain layers, components or features may be exaggerated for clarity, and broken lines illustrate optional features or operations unless specified otherwise. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The terminology used herein is for the purpose of describing particular embodiments only and is not intended to be limiting of the invention. As used herein, the singular forms "a", "an" and "the" are intended to include the plural forms as well, unless the context clearly indicates otherwise. It will be further understood that the terms "comprises" and/or "comprising", when used in this specification, specify the presence of stated features, integers, steps, operations, elements, and/or components, but do not preclude the presence or addition of one or more other features, integers, steps, operations, elements, components, and/or groups thereof. As used herein, the term "and/or" includes any and all combinations of one or more of the associated listed items.

As used herein, phrases such as "between X and Y" and "between about X and Y" should be interpreted to include X and Y. As used herein, phrases such as "between about X and Y" mean "between about X and about Y." As used herein, phrases such as "from about X to Y" mean "from about X to about Y."

It is noted that aspects of the invention described with respect to one embodiment, may be incorporated in a different embodiment although not specifically described relative thereto. That is, all embodiments and/or features of any embodiment can be combined in any way and/or combination. Objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

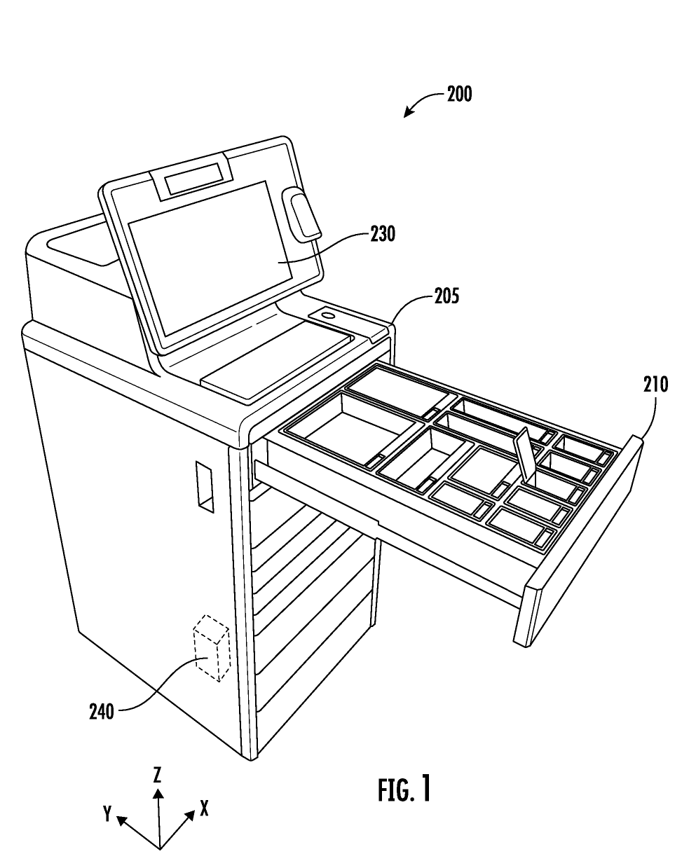

Referring now to the figures, FIG. 1 illustrates an automated dispensing machine (ADM) for pharmaceuticals, designated broadly at 200, that may be used in a hospital and other health care settings. The machine 200 includes a generally box-shaped cabinet 205 with a number of vertically-stacked drawers 210 that are slidable relative to the remainder of the cabinet 205. Herein drawers 210 refers to drawers of any number of possible embodiments with specific embodiments depicted in the figures being referred to as 210a, 210b, etc.

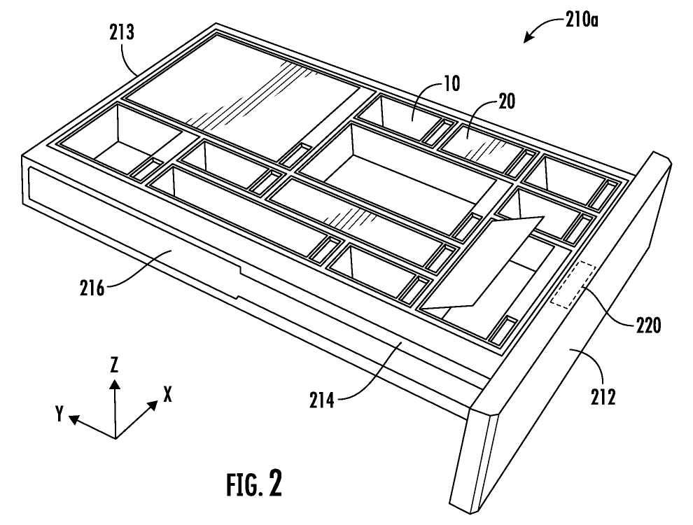

As seen in FIG. 2, in some embodiments, each of the drawers 210a is locked in a closed position via a drawer locking mechanism 220 that prevents access to the drawer 210a without permission. In such embodiments, either the drawer 210a, the drawer locking mechanism 220, or both are operatively connected with a main controller 240 in the machine 200 that prevents the drawer 210a from opening without access having been granted in the manner described below.

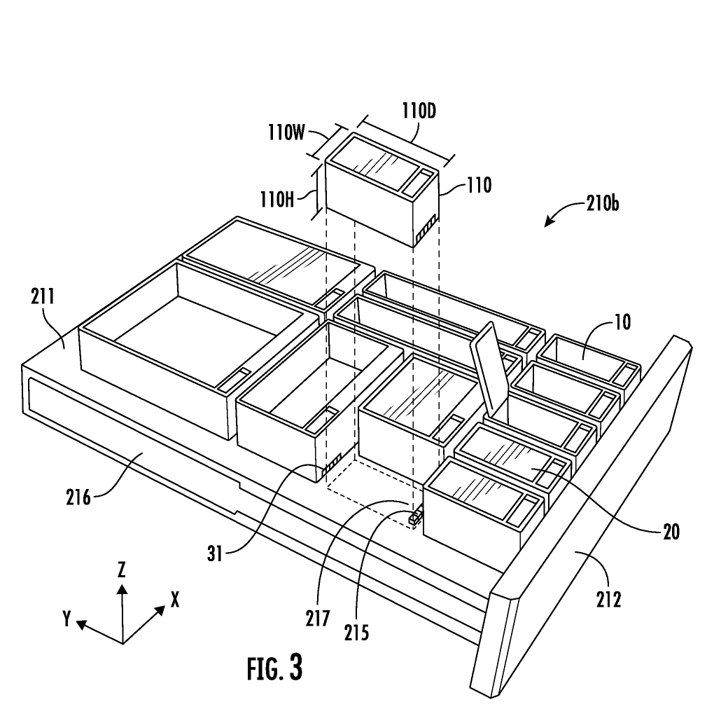

FIGS. 2 and 3 illustrate exemplary drawers 210. Each drawer 210 includes at least a floor 211 and a front wall 212. In some embodiments, each drawer 210 also includes a back wall 213 and side walls 214 forming a hollow rectangular prism with an open top. In some embodiments, the drawer 210 may include sliding mechanisms 216 which permit the drawer 210 to slide in and out of the machine 200. In some embodiments, the sliding mechanisms 216 are mounted to the side walls 214. In further embodiments, the sliding mechanisms 216 are mounted to the floor 211 as shown in the drawer 210b shown in FIG. 3. The drawers 210 may be configured to be detachably mounted to the machine 200. In other words, the drawers 210 may be configured such that it can be disconnected and removed from the machine 200. In some embodiments, the machine 200 may contain drawers 210 of varying heights. For example, the machine 200 a first drawer with a first height and a second drawer with a second height. The second height may be approximately half the distance of the first height.

Within each drawer 210 are either open containers 10, lidded containers 20, or a mixture of both, each of which can contain a bulk supply of a medicament or other medical item (e.g., a bandage, syringe, or the like). Herein containers 10, 20, open containers 10, and lidded containers 20 refer to containers of any number of possible embodiments with specific embodiments depicted in the figures being referred to as 10a, 10b, 20a, 20b, etc. Each of the containers 10, 20 may be located in a drawer location 217. The main controller 240 may be configured to record which containers 10, 20 are in which drawer locations 217. In some embodiments, the drawer location 217 is understood by the main controller 240 as the surface area of the floor 211 of the drawer 210 covered by the container 10, 20. The drawer location 217 may also be understood by the main controller 240 as the approximate location of a connector 215 engaged by the container 10, 20. The open and lidded containers 10, 20 may include a vial or bottle containing a bulk supply of pills, tablets, etc., or the open and lidded containers 10, 20 may include the pills or other items loosely contained within the open and lidded containers 10, 20; in some instances the loosely contained pills or other items may be individually packaged. The drawer 210 may include space not utilized or intended for containers 10, 20. For example, the internal area of the drawer 210 may be approximately half full of containers 10, 20 and approximately half unoccupied space. The unoccupied space may be discontinuous, a front portion (such as a front half), a back portion (such as a back half), or a side portion (such as a side half).

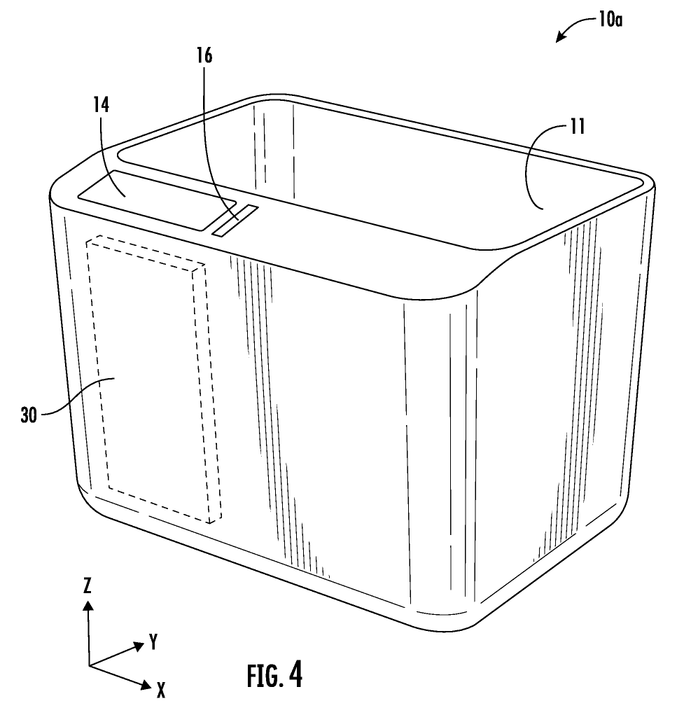

FIGS. 4 through 9 illustrate exemplary open and lidded containers 10, 20. Each of the open containers 10 is a hollow rectangular prism which comprises an internal cavity 11 and are both open-topped and lidless to permit swift access to a user (e.g., a medical professional such as a doctor, nurse, or medical technician) at any time. The open containers 10 may be used for holding items that require lower levels of security such as bandages, over-the-counter medications, tongue depressors, cotton swabs, etc.

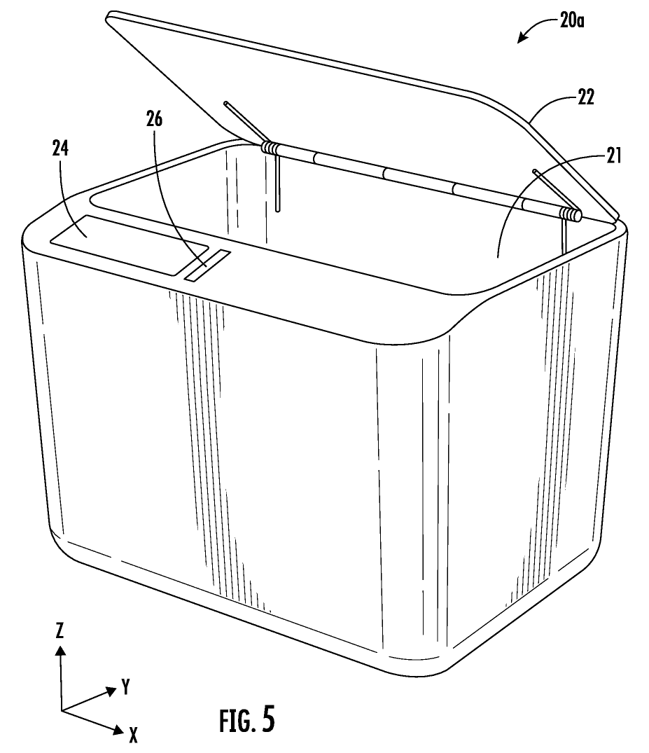

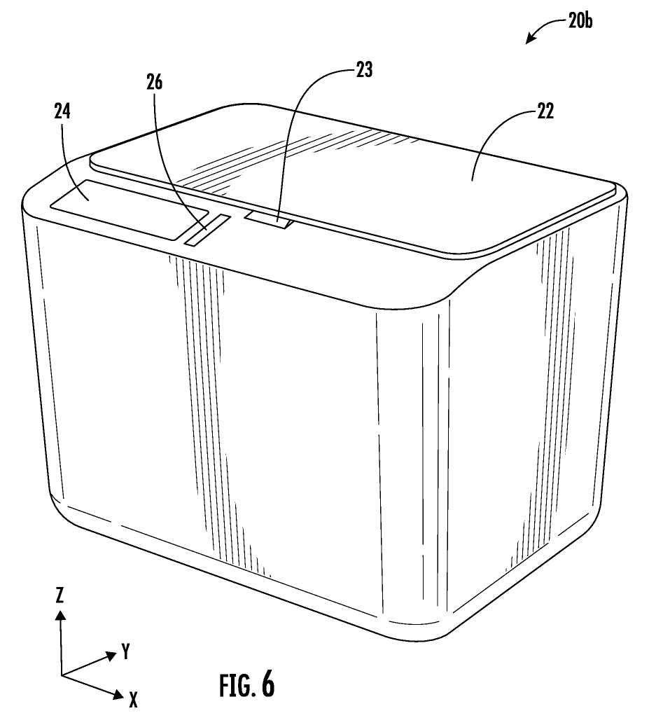

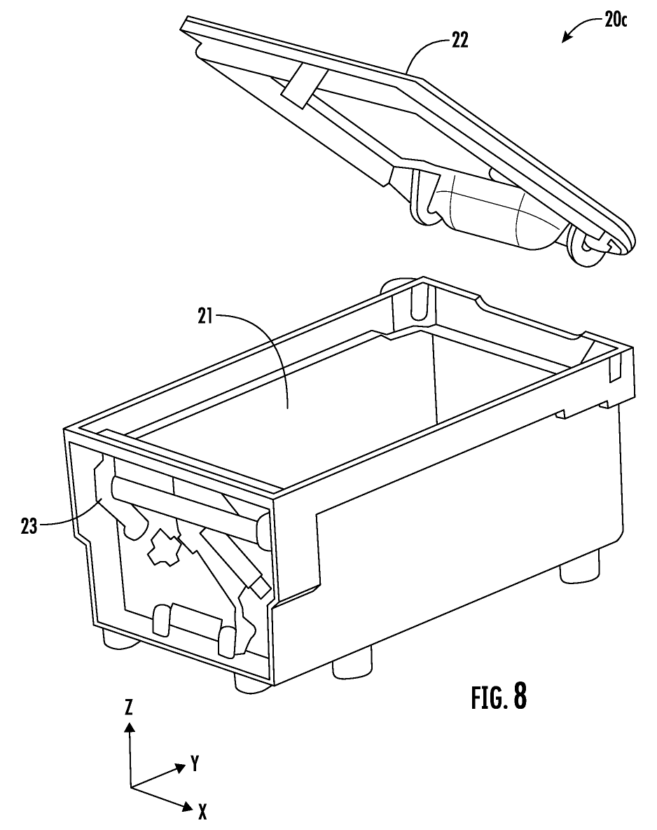

As is shown by the lidded container 20a depicted in FIGS. 5, 6, and 8, each of the lidded containers 20 comprises an internal cavity 21 and a lid 22, They may be locked via a container locking mechanism 23 to prevent access to a user (e.g., a medical professional such as a nurse or medical technician) unless access is permitted in the manner described below. Exemplary lidded containers 20b, 20c with container locking mechanisms 23 are depicted in FIGS. 6 and 8; however, a person of ordinary skill in the art will recognize that many known container locking mechanisms could be utilized. Typically, each lidded container 20 is configured such that its lid 22 pivots to an open position to provide access to the internal cavity 21 of the lidded container 20 where the medicaments or other medical items reside. In some alternative embodiments, each lidded container 20 is configured such that its lid 22 slides to an open position instead of pivoting. Exemplary container locking mechanisms for the drawers 210 and lidded containers 20 are shown, for example, in U.S. Patent Nos. 10,029,829 and 10,435,918, supra. An exemplary machine 200 with such drawers 210 and lidded containers 20 is the Pyxis™ system, available from Becton, Dickinson & Company (Franklin Lakes, New Jersey).

As is shown by the open container 10a depicted in FIG. 4, in some embodiments, each open and lidded container 10, 20 includes a local controller 30 (e.g., a printed circuit board (PCB)) that interfaces with the main controller 240. In further embodiments, the main controller 240 and the local controller 30 are the same component or are both subcomponents of an overall controller system. Typically, the interface connection between the open or lidded container 10, 20 and the main controller 240 is established when the open or lidded container 10, 20 is installed in the drawer 210.

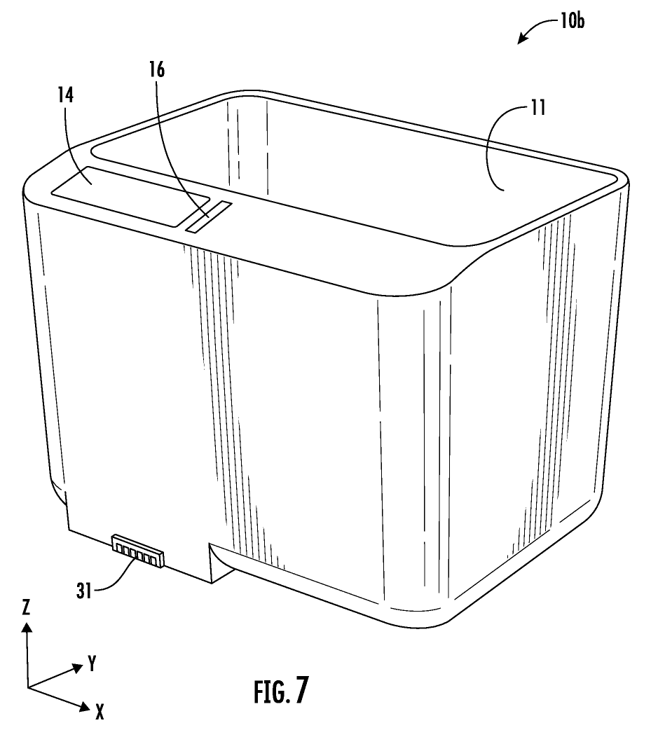

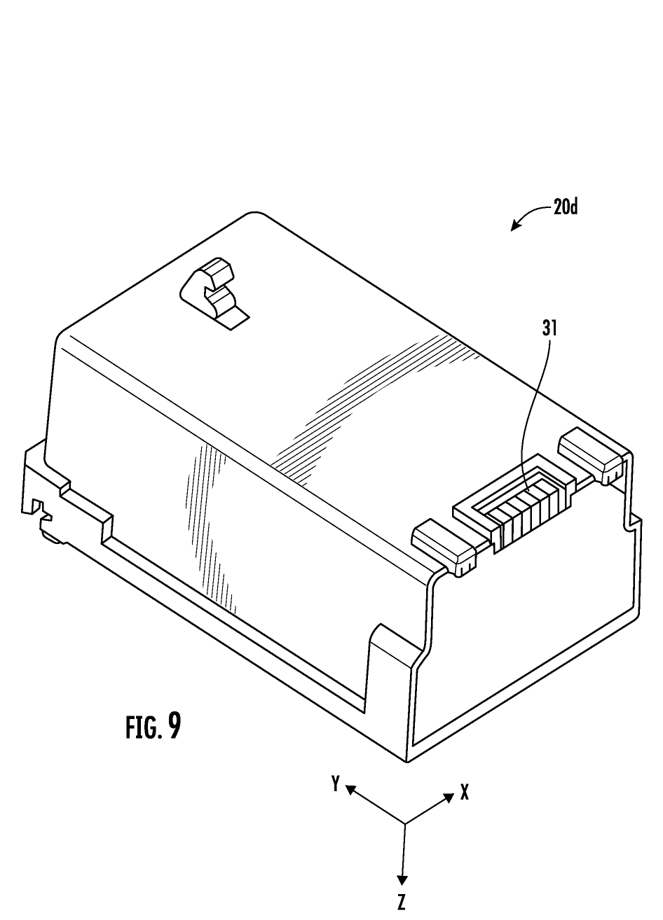

One of ordinary skill in the art will recognize that there are many suitable methods for installing the open and lidded containers 10, 20 into the drawer 210. As is shown by the open container 10b depicted in FIGS. 7 and 9, in some embodiments the containers 10, 20 may include a port 31 which may be connected to and/or a part of a local controller 30. The port 31 may mate with a connector 215, such as a plug or socket, in the drawer 210 that is connected to the main controller 240. The connector 215 may be on or within any surface of the drawer 210 including the top surface of the floor 211. In some embodiments, wires (not shown) are routed from the local controllers 30 of the open and lidded containers 10, 20 to their respective connectors 215 in the drawer 210. In some further embodiments, the local controllers 30 connect directly to the connectors 215. Exemplary interface connections are discussed in, for example, U.S. Patent No. 9,687,075, supra.

In some embodiments, an additional frame (not shown) is installed within the drawer 210 and the open and lidded containers 10, 20 are installed into the frame (not shown). In such embodiments, the frame (not shown) may operatively serve as an intermediary with the open and lidded containers 10, 20 connecting to the frame (not shown) and the frame (not shown) connecting to the connector 215 of the drawer 210. However, in other embodiments, the open and lidded containers 10, 20 may still connect directly to the drawer 210 as described hereinabove even if they are first installed within a frame (now shown). Additionally features of the containers 10, 20 discussed herein, such as additional lights or labels, may be a feature of a frame (not shown) instead. One of ordinary skill in the art will recognize that such a frame (not shown) could be designed in any number of ways.

A user interface 230 (represented herein by a display monitor and/or a keyboard) is operatively connected with the drawer locking mechanisms 220 and with container locking mechanisms 23 of the lidded containers 20. The user interface 230 is operatively connected with the main controller 240 that is, as described above, operatively connected with the drawers 210, the open containers 10, and the lidded containers 20. The main controller 240 is configured to enable the user to selectively open a specific lidded container 20 via inputs into the user interface 230 while the rest of the lidded containers 20 remain inaccessible. In some embodiments, the main controller 240 is configured to enable a user to selectively open one of the drawers 210 via data input via the user interface 230 (typically related to the identity of the user and a specific patient or group of patients). In some further embodiments, the drawers 210 are configured so that the drawer 210 of interest slides open automatically upon the correct input from the technician. Also, in some embodiments, the lidded containers 20 are configured so that the lidded container 20 that contains the medicament or medical item of interest opens automatically once the drawer 210 is opened. In other words, when a user inputs the correct information into the user interface 230 to qualify for permission to access the medicament or medical item, the main controller 240 signals the correct drawer 210 and correct lidded container 20 to open (i.e., to unlock their respective locking mechanisms) to provide access to the contents of that lidded container 20 to the user. As this occurs, access to other lidded containers 20 within that drawer 210 is typically denied unless the input provided by the technician also granted permission to more than one lidded container 20. Access to open containers 10 is typically permitted at all times. In embodiments where the drawers 210 lock, access to open containers 10 may be limited until a user selectively opens one of the drawers 210 via data input via the user interface 230.

In some embodiments, the main controller 240 will be operatively connected with an external device, such as a personal or mainframe computer, that provides input information regarding prescriptions, patients, medical personnel that may access the containers (e.g., locally or over a network). In other embodiments, the main controller 240 may be a stand-alone computer that directly receives manual input from a pharmacist or other operator through the user interface 230. An exemplary main controller 240 is a conventional microprocessor-based personal computer. The main controller 240 may also be divided into multiple computers, networks, processors, etc., that combine and/or share operations. For example, one portion of the main controller 240 may be internal to the machine 200 and control certain operations of the machine 200, and another portion of the main controller 240 may be external to the machine 200 and control other operations of the machine 200. Alternatively, the user interface may be or include a handheld device.

Typically, the open and lidded containers 10, 20 installed in the drawer 210 are similar in size and function to the others of the open and lidded containers 10, 20, and therefore are potentially interchangeable with each other, which enables the machine 200 to be set up as needed (e.g., if one medicament experiences heavy usage, additional containers 10, 20 of that medicament may be included in the machine 200 or a larger version of a container 10, 20 may be used for that medicament).

In some embodiments, open and lidded containers 10, 20 of varying sizes may be used in the same drawer. The open and lidded containers 10, 20 of varying sizes may have a uniform sizing scheme wherein their dimensions in each direction (i.e., width, height, and depth) are approximately multiples of those of a base container 110. For example, the base container 110 may have a one base width 110W in an X-direction, one base depth 110D in a Y-direction, and one base height 110H in a Z-direction. Typically, this uniform sizing scheme is utilized both for ease of configuration and aesthetic appeal; however, in some embodiments containers of nonuniform sizes may be utilized. Furthermore, a uniform sizing scheme increases the ease with which a drawer layout can be chosen. Only a finite number of solutions are possible and any “holes” in a desired configuration can easily be filled with as many base containers 110 as necessary. A uniform sizing scheme also simplifies the layout of connectors 215 within the drawer 210 by allowing the drawer to contain a grid system of connectors each at the same height in a Z-direction and separated by one base width 110W in an X-direction and one base depth 110D in a Y-direction. In some embodiments, each of the containers 10, 20 may be the base height 110H in the Z-direction and the base height 110H may be less than the height of the internal area of the drawer 210. For example, the base height 110H may be approximately half of the height of the internal area of the drawer 210.

When a container 10, 20 is larger than the base container 110, for example a container 10, 20 which is two base depths 110D in depth, it may cover more than one connector 215. The containers 10, 20 may be configured to engage one, some, or all of the connectors 215 that it covers. In some embodiments, the containers 10, 20 may further comprise connector caps (not shown) configured to engage the open or unused connectors 215. The connector caps (not shown) may be configured to cover, but not engage, the open or unused connectors 215 and may be configured to prevent damage to the open or unused connectors 215.

In some embodiments, the open and lidded containers 10, 20 may be shapes other than rectangular prisms. As an example, an open or lidded container 10, 20 may be shaped roughly like a capital letter “L”, wherein the “L” has a vertical shaft and horizontal leg, the vertical shaft has three base depths 110D in the Y-direction and one base width 110W in the X-direction, and the horizontal leg has one standard depth 110D in the Y direction and one base width 110W in the x direction. Such configurations allow for the storage of oddly-shaped objects.

The open and lidded containers 10, 20 may be detachably mounted within the drawer 210. Detachably mounting the open and lidded containers 10, 20 may allow for modifying the drawer 210 with new configurations and/or different sizes and shapes of containers. Exemplary detachably mounted containers are discussed in, for example, U.S. Patent No. 9,687,075, supra. In some embodiments, the main controller 240 and/or user interface 230 is capable of generating suggested configurations based on input data from the user concerning the preferred shapes and sizes of some containers to be included in the configuration.

The machine 200 may be further connected to a larger network of similar storage machines, each of these similar storage machines comprising additional drawers and open and lidded containers. In such a network, each of the open and lidded containers 10, 20 may be configured to record where it is located. In other words, each of the open and lidded containers 10, 20 may be configured to record which machine 200, drawer 210, and/or drawer location 217 it is in. Each of the open and lidded containers 10, 20 may then be further configured to communicate this information to each main controller 240 in the network. In some embodiments, the main controller 240 of each machine 200 is further configured to record the floor plan of the building or complex it is within, the location of each machine 200 within the same network, the location of each networked drawer 210, the location of each networked open and lidded container 10, 20, and the contents of each networked open and lidded container 10, 20. This information may then be available to the user through the user interface 230. The main controller 240 may be further capable of directing a user to a specific location of a specific item through the user interface 230. Exemplary systems for distributed medication management are shown, for example, in U.S. Patent Nos. 11,348,673 and 12,033,735.

In some embodiments, the main controller 240 or local controller 30 may also track the inventory levels of some or all of the open and/or lidded containers 10, 20. The inventory tracking may be achieved through any number of means. For example, each of the open and/or lidded containers 10, 20 may be equipped with a sensor which senses access to the open and/or lidded containers 10, 20. Such a sensor may be, for example, a pressure plate, a camera, a motion sensor, or a photoelectric sensor; however, the sensors of the present inventive concept should not be limited thereto. The lidded containers 20 may also be configured to sense when their lid is opened. The main controller 240 or local controller 30 may be configured to request information from the user concerning the quantity of removed or added stock. Information on stock removals or additions may be used by the main controller 240 or local controller 30 in concert with information provided by a sensor to track inventory levels or may be the primary method of monitoring inventory.

The main controller 240 or local controller may also be configured to attempt to automatically detect changes in inventory level. In some instances, the open and lidded containers 10, 20 may be equipped with a sensor which senses a change in inventory level. Such a sensor may be, for example, a pressure plate or a camera; however, the sensors of the present inventive concept should not be limited thereto. In some embodiments, a pressure plate sensor may be configured to approximate the inventory level of an open and/or lidded container 10, 20 based on the measured weight of the open and/or lidded container’s 10, 20 contents and the known average weight of the items in the open and/or lidded container 10, 20. In some embodiments, a camera may observe the removal of contents from an open and/or lidded container 10, 20. The main controller 240 or local controller 30 may be configured to approximate the quantity of inventory removed based on the observations of the camera.

The open and lidded containers 10, 20 can be individually labeled. In some embodiments, the label 14, 24 may be physically part of the open or lidded container 10, 20 (such as being etched into the surface of the open or lidded container 10, 20 or being raised from them), may be written or printed directly on the open or lidded container 10, 20, or may be printed on a sticker affixed to the open or lidded container 10, 20. Typically the label 14, 24 will be an electronic e-ink label. Exemplary e-ink products are available from E Ink Corporation, Billerica, Massachusetts and are discussed in, for example, U.S. Patent No. 12,158,684. Exemplary uses of e-ink in the context of medical or medication storage are discussed in, for example, U.S. Patent No. 12,033,735, supra.

The label 14, 24 may include information concerning the open and lidded container 10, 20 itself as well as its contents. In some embodiments, this information includes, but is not limited to, the name of the contents within the container, the dosage of any medication, expiration date, inventory level, a barcode, a serial number etc.

In addition to the e-ink label, other features may be implemented to direct users to a specific open or lidded container 10, 20. In some embodiments, the lid 22 may be configured to open in response to a user interaction with the user interface 230. The lid or lids 22 of the desired lidded containers 20 are typically the only lids 22 open and may, therefore, serve to direct the user to the specific lidded container 20 containing the desired items.

The containers 10, 20, drawer 210, and/or another part of the machine 200 may include one or more indicators for communicating information to a user. The indicators may be, for example, lights 16, 26 configured to indicate to a user whether a container 10, 20 has items desired by the user In other words, the open containers 10, the lidded containers 20, or both comprise at least one light 16, 26. In response to user input via the user interface 230, the main controller 240 may utilize these lights 16, 26 to direct the user to a specific open or lidded container 10, 20 containing the items desired by the user. Such lights 16, 26 may be configured to shine a single color or in multiple colors to indicate various information to the user. For instance, an off system may be implemented wherein the lights 16, 26 of the majority of open and lidded containers 10, 20 are off and only the lights of the desired container(s) are on. Alternatively, a green/red system may be implemented wherein the lights 16, 26 may shine green to indicate which container 10, 20 is the desired container and the remaining lights 16, 26 may shine red to indicate the container 10, 20 they are on is not the desired container. The lights 16, 26 may be LED lights, but are not limited thereto.

In some embodiments the lights 16, 26 are utilized to provide information other than the location of a desired container. For example, a green/yellow/red system may be implemented to indicate inventory levels of the contents of the open and lidded containers 10, 20. In such embodiments, green may indicate a high or sufficient inventory, yellow may indicate a moderate or low inventory, and red may indicate a low or exhausted inventory. In some embodiments, the lights 16, 26 may be configured to indicate that a lidded container 20 which has failed to open was intended to open.

In some embodiments the lights 16, 26 may be a single light for each open and lidded container 10, 20. In other embodiments, each open and lidded container 10, 20 may include multiple lights 16, 26. These lights 16, 26 may be located anywhere on or near the open or lidded containers 10, 20 they are intended to be associated with. In some embodiments a series of lights surround the open side of the internal cavity 11, 21.

The user interface 230 may also direct users to a desired open or lidded container 10, 20. The user interface 230 may be configured to directly communicate to the user which open or lidded container 10, 20 contains a desired item and where that open or lidded container 10, 20 is located. This information may be communicated visually (for example, on a screen), audibly, through touch (for example, braille), or by a combination thereof.

The drawer 210 may further comprise containers than are neither lidded nor open containers 10, 20 as described herein. Such containers may be entirely enclosed with an automatic dispensing system. Exemplary automatic dispensing containers are discussed in, for example, U.S. Provisional Patent Application No. 63/677,484.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention.

What is claimed is:

1. A cabinet for holding and dispensing items comprising:
a housing including a cavity;
at least one drawer mounted in the cavity, the drawer comprising;
at least one open container mounted in the drawer with an internal compartment and an open upper end; and
at least one lidded container mounted in the drawer with an internal compartment and a lid;
wherein the lid is attached to the at least one lidded container via a lid mechanism, the lid mechanism being configured to move the lid between an open position and a closed position; and
a controller operatively connected to the lid mechanism.

2. The cabinet of claim 1, wherein the lid mechanism is configured such that the lid of the at least one lidded container remains locked in the closed position unless the controller provides a signal indicating permission to access.

3. The cabinet of claim 1, wherein the lid mechanism is configured such that the lid automatically moves to the open position when the controller provides a signal indicating permission to access.

4. The cabinet of claim 1, wherein the at least one open container and the at least one lidded container are detachably mounted to the drawer.

5. The cabinet of claim 1, wherein the at least one drawer is configured such that it can be disconnected and removed from the housing.

6. The cabinet of claim 1, wherein the at least one open container further comprises an e-ink label; and
the at least one lidded container further comprises an e-ink label.

7. The cabinet of claim 1, wherein the at least one open container further comprises a first indicator configured to indicate to a user whether the at least one open container has items desired by the user; and
the at least one lidded container further comprises a second indicator configured to indicate to a user whether the at least one lidded container has items desired by the user.

8. The cabinet of claim 1, wherein the at least one open container has a first depth and the at least one lidded container has a second depth the differs from the first depth.

9. The cabinet of claim 1, wherein each of the at least one open container and the at least one lidded container has a sensor, wherein the sensor is configured to sense when the at least one open container or the at least one lidded container is accessed by a user and wherein the sensor is further configured to send a signal to the controller when the at least one open container or the at least one lidded container is accessed by the user.

10. A drawer for a cabinet for holding and dispensing items comprising:
a frame including a floor and front wall;
at least one open container mounted in the drawer with an internal compartment, an open upper end, and a floor;
at least one lidded container mounted in the drawer with an internal compartment, a lid, and a floor;
wherein the lid is attached to the lidded container via a lid mechanism; and
a controller operatively connected to the lid mechanism;
wherein the floors of each of the at least one open container and the at least one lidded container are substantially coplanar.

11. The drawer of claim 10, wherein the lid mechanism is configured such that the lid of the at least one lidded container remains locked in the closed position unless the controller provides a signal indicating permission to access.

12. The drawer of claim 10, wherein the lid mechanism is configured such that the lid automatically moves to the open position when the controller provides a signal indicating permission to access.

13. The drawer of claim 10, wherein the at least one open container and the at least one lidded container are detachably mounted to the drawer.

14. The drawer of claim 10, wherein the at least one drawer is configured such that it can be inserted and connected to a cabinet.

15. The drawer of claim 10, wherein the at least one open container further comprises an e-ink label; and
the at least one lidded container further comprises an e-ink label.

16. The drawer of claim 10, wherein the at least one open container further comprises a first indicator configured to indicate to a user whether the at least one open container has items desired by the user; and
the at least one lidded container further comprises a second indicator configured to indicate to a user whether the at least one lidded container has items desired by the user.

17. The drawer of claim 10, wherein the at least one open container has a first depth and the at least one lidded container has a second depth the differs from the first depth.

18. The drawer of claim 10, wherein each of the at least one open container and the at least one lidded container has a sensor, wherein the sensor is configured to sense when the at least one open container or the at least one lidded container is accessed by a user and wherein the sensor is further configured to send a signal to the controller when the at least one open container or the at least one lidded container is accessed by the user.

19. A cabinet for holding and dispensing items comprising:
a housing including a cavity;
at least one drawer mounted in the cavity, the drawer comprising;
at least one open container mounted in the drawer with an internal compartment, a local controller, and an open upper end;
wherein no containers in the drawer are lidded containers; and
a main controller operatively connected to the at least one drawer.

20. The cabinet of claim 19, wherein the at least one open container further comprises a first indicator configured to indicate to a user whether the at least one open container has items desired by the user.
